(19) United States
(12) Patent Application Publication
(10) Pub. No.: US 2004/0248101 A1
(43) Pub. Date: Dec. 9, 2004

(54) Identification of novel antibacteria agents by screening the single-stranded DNA expression library

(75) Inventors: Yin Chen, Pearland, TX (US); Xin Xing Tan, Manvel, TX (US)

Correspondence Address: Wisner & Associates, Suite 400, 1177 West Loop South, Houston, TX, 77027-9012, US

(73) Assignee: CytoGenix, Inc., Houston, TX (US)

(21) Appl. No.: 10/453,410

(22) Filed: Jun. 3, 2003

(51) Int. Cl. C12Q 1/68; C07H 21/04; C12N 15/74; C12N 1/21

(52) U.S. Cl. 435/6; 536/23.1; 435/472; 435/252.3

(57) ABSTRACT

A selectively inducible, single-stranded DNA (ssDNA) expression library, a method for constructing a ssDNA expression library, a method for screening ssDNA using the expression library, and a method for identifying ssDNA molecules that switch on or off bacterial gene(s) related to cell growth and toxin production and secretion. The screening library is used to, among other things, identify ODNs effective in stopping bacterial growth, killing bacteria or preventing bacteria from synthesizing and secreting their toxins is the focus of the present invention and/or to discover ODNs effective in eukaryotic (e.g., mammalian) cells for targeted gene down regulation. The library is also useful for identifying ssDNAs or ODNs that are used as therapeutic antibacterial reagents, for identifying essential bacterial genes that can serve as targets for antibiotic discovery, and for providing a method for treatment of bacterial infections.

### BACKGROUND OF THE INVENTION

Oligonucleotide-mediated intervention (OMI) technology provides a powerful set of tools to alter the activity of any gene of known sequence. The ability to produce single strands of DNA (ssDNA) of any sequence and length in selected cells enables targeted alteration of gene expression at the genomic level using triplex forming oligonucleotides for targeted gene expression, at the messenger RNA (mRNA) level using antisense and DNA enzyme oligos and at the protein level using ssDNA as aptamers (Chen, Y. 2002, Expert Opin. Biol. Ther. 2(7) 735-740).

Antisense, DNA enzyme, triplex, and aptamer technologies provide an efficient alternative to more difficult methods such as creating gene knockout in cells and organisms. Antisense oligonucleotides (ODNs) block gene expression by Watson-Crick base pairing between an ODN and its target mRNA (Crooke, S. T. 1999, Biochim. Biophys. Acta 1489:31-44). Antisense ODNs have been used to effectively inhibit gene expression in eukaryotic cells and have been used to validate gene targets. There is one antisense ODN-based product in the market and a number of others in advanced clinical trials (Uhlman, E., 2001, Expert Opinion on Biological Therapy, 1:319-328). However, antisense technology is not used extensively in prokaryotic systems. Prokaryotic cells have themselves developed endogenous antisense mechanisms for gene regulation (Simons & Kleckner, 1988, Annu. Rev. Genet., 22, 567-600). Earlier results indicated that gene expression in bacteria may be accessible to inhibition by modified ODNs (Jayayaraman, et al., 1981, PNAS, 78:1537-1541; Gasparro, F. P., et al., 1991, Antisense Res Dev., 1:117-140). Others reported that peptide nucleic acid (PNA) can inhibit gene expression in bacteria. (Good & Nielsen, 1998, Nature Biotechnology, 16:355-358). PNA, a DNA mimic in which the nucleotide bases are attached to a pseudopeptide backbone, hybridizes with complementary DNA, RNA, or PNA oligomers through Watson-Crick base pairing and helix formation.

One major parameter determining efficacy of any OMI strategy is target site accessibility. The lack of effectiveness of antisense or other ODNs may largely be a result of selecting inaccessible sites in the target. Undoubtedly, base composition can affect heteroduplex formation. However, it does not appear to be the primary factor. There is now convincing evidence that binding of complementary ODNs is mainly determined by the secondary and tertiary structures of RNA molecules (Frauendorf A., et al., Bioorg. Med. Chem. Lett., 1996, 4:1019-1024).

Various approaches to identifying the accessible sites on target mRNAs in relation to antisense and/or DNA enzyme design have been developed. Conventionally, a linear shot-gun approach has been used to select antisense ODNs. Several oligonucleotides, targeted to various region of an mRNA, are synthesized individually and their antisense, DNA enzymatic or other activity (or binding affinity to the target sites) measured. However, only 2-5% of ODNs are generally found to be good antisense reagents.

In an attempt to introduce rationality and efficiency into efforts to identify active OMI reagents, researchers also use computer programs. For instance, the secondary structure of target RNA is predicted using an RNA folding program, such as mfold (M. Zuker, 1989, Science, 244, 48-32). Antisense ODNs are designed to bind to regions that are predicted to be free from intramolecular base pairing. However, energy-based prediction methods of RNA structure are largely inadequate for designing antisense reagents and success using this approach has been limited.

Evidence that ribonuclease H (RNase H) is involved in antisense-mediated effects has led to the development of several procedures that make use of this enzyme to identify accessible binding sites in mRNAs in vitro. RNase H is an endoribonuclease that specifically hydrolyzes phosphodiester bonds of RNA in DNA:RNA hybrids. RNase H may be used in combination with a random ODN library comprising a complete set of all possible ODNs of a defined length. For instance, for a length N, there are thus N⁴ different possible ODNs in the library set such that there would be approximately 2.56×10⁶ molecules for a 40-mer ODN. Component ODNs of the library that are complementary to accessible sites on the target RNA produce hybrids with RNA that are identified as RNase H cleavage sites by gel electrophoresis. While many of the possible ODNs in the library set are of no interest; e.g., an ODN such as AAAA . . . AAAA, is useful to test the library set members to see which, if any, produces a down regulating effect on a specific target mRNA. Controlled gene expression systems such as the tetracycline regulatory system in prokaryotic cells allow selective gene down or up-regulation and thereby supply information on the gene product.

Hammerhead and hairpin ribozymes are catalytic RNA molecules that bind defined RNA targets and enzymatically cleave RNA targets and have been used successfully to knock down gene expression of viral and cellular targets (for review, see James, H. A. & Gibson, I., Blood, 91:37i-382, 1998). Pierce and Ruffner have successfully developed a method to identify accessible sites on the ICP4 mRNAs for antisense-mediated gene inhibition using a hammerhead ribozyme library that allows expression of the library components in mammalian cells (Pierce & Ruffner, 1998, Nucleic Acid Research, 26:5093-5101). ICP4 is an essential transcriptional activator of the Herpes simplex virus (HSV). Although hammerhead ribozymes can efficiently cleave specific mRNA targets, clinical application is limited because of instability caused by RNase degradation in vivo.

Identifying a gene or gene family responsible for a particular phenotype is crucial to the deciphering of any biological mechanism and our understanding of disease. Ribozyme libraries can be used not only to identify accessible sites on target mRNA, but also genes that are directly involved in producing a particular phenotype. Researchers from Immusol, Inc. constructed a hairpin ribozyme library that was delivered to mammalian cells either with plasmid or retroviral vectors (Welch, P. J. et al., Genomics, 66, 274-283, 2000, Li, Q., et al., Nucleic Acid Research, 28:2605-2612, 2000, Kruger, M., et al., PNAS, 97:8566-8571, 2000, Beger, C., et al., PNAS, 98:130-135, 2001). By knocking-down or knocking-out gene expression using a ribozyme library, they were able to identify novel gene or new functions of known genes such as 1) the human homologue of the Drosophila gene ppan, involved in mammalian cell growth control 2) telomerase reverse transcriptase (mTERT), a suppressor of cell transformation; 3) eukaryotic translation initiation factors, eIF2Bγ and eIF2γ, as cofactors of hepatitis C virus internal ribosome entry site-mediated translation; and 4) transcriptional regulator, Id4, as a regulator of BRCA1 gene expression. However, similar to hammerhead ribozymes, hairpin ribozymes have limited stability in vivo.

Ji, et al. constructed a library of small staphylococcal DNA fragments (200 to 800 bp) derived by shearing genomic DNA (Ji, et al., 2001, Science, 293:2266-2269). By transforming the library into Staphylococcus aureus, random antisense RNA molecules were generated. Using this approach, Ji, et al. identified critical genes that could serve as targets for antibiotic discovery. A similar approach has been used by Forsyth, et al. in S. aureus (Forsyth, et al., 2002, Molecular Microbiology, 43:1387-1400). However, this approach can only be used for the identification of essential genes since antisense RNA with the size between 200-800 bp is not useful for therapeutic purposes because of 1) the instability of RNA molecules; 2) the difficulty of synthesizing RNA molecules with the size of 200-800 bp; and 3) the problem of delivering RNA to appropriate cells.

Traditional antibiotics are low-molecular-weight compounds that either kill (bactericidal) or inhibit (bacteriostatic) the growth of bacteria. Most of them are produced by microorganisms, especially Streptomyces spp. and fungi. Antibiotics are directed against targets that are preferably specific to bacteria, which minimize the potential toxicity of the antibiotic in humans. Specific targets include inhibitors of cell wall biosynthesis, aromatic amino acid biosynthesis, cell division, two component signal transduction, fatty acid biosynthesis, isopreniod biosynthesis and tRNA synthesis. For example: 1) Penicillin blocks the final step of cell wall synthesis by binding covalently to the active site of the tranpepetidase enzyme; 2) Kanamycin inhibits protein synthesis by interacting with bacterial ribosomal 30S RNA; 3) Rifampicin binds to the s subunit of bacterial RNA polymerase, the enzyme required to transcribe mRNA from the bacteria DNA; 4) Trimethoprim, which is a bacterial dihydrofolate reductase inhibitor while leaving the mammalian enzyme virtually unaffected; and 5) Ciprofloxacin, which inhibits bacterial toposiomerase II or DNA gyrase, the enzyme that controls the supercoiling or folding of the bacterial chromosome DNA within the cells. Inhibitors in categories 4) and 5) are not traditional antibiotics since they are completely synthetic compounds.

In recent years, there has been a rapid emergence of antibiotic resistance to many common bacterial pathogens such as S. aureus, Streptococcus pneumoniae and Enterococcus faecalis (Nicolaou, K. C. & Boddy, C. N. C., 2001, Scientific American, p.56-61). Methicillin-resistant S. aureus (MRSA), penicillin-resistant S. pneumococcus and vancomycin-resistant E. faecalis (VRE) are now common pathogens that are difficult to treat effectively (Pfaller, M. A., et al., 1998, Antimicrobiol Agents and Chemotherapy, 42:1762-1770; Jones, R. N., et al., 1999, Microbiology and Infections Disease, 33:101-112). Probably more alarming is the emergence of multi-drug resistance pathogens (Swartz, M. N., 1994, PNAS, 91:2420-2427; Baquero, F., 1997, J. Antimicrobial Chemotherapy, 39:1-6). Until recently, the principal approach of the pharmaceutical industry has been to seek incremental improvements in existing drugs. Although these approaches have made a significant contribution to combating bacterial infections, they are having difficulty meeting the increasing needs of the medical community. Health care workers are increasingly finding that nearly every weapon in their arsenal of more than 150 antibiotics is becoming useless. Infectious diseases such as tuberculosis, meningitis and pneumonia, that would have been easily treated with antibiotics at one time, are no longer so readily thwarted.

There is, therefore, an emergent demand for the discovery and development of new classes of antibiotics to add to the current arsenal. Recent advances in DNA sequencing technology have made it possible to elucidate the entire genome sequences of pathogenic bacteria. Gernomic sequencing reveals all of the information in bacteria related to potential targets by antibiotics and therefore provides a more rational target-based approach to develop new antibiotics.

The use of a screening library to identify ODNs effective in stopping bacterial growth, killing bacteria or preventing bacteria from synthesizing and secreting their toxins is the focus of the present invention. Use of the screening library to discover ODNs effective in eukaryotic (e.g., mammalian) cells for targeted alteration of gene function is a logical application.

It is, therefore, an object of the present invention to provide a method for identifying ssDNAs or ODNs that are used as therapeutic antibacterial reagents.

An additional object of the present invention is to provide a method for identifying essential bacteria genes that can serve as targets for antibiotic discovery.

An additional object of the present invention is the provision of a method for the treatment of bacterial infections.

An additional object of present invention is to provide a method for the regulation of gene expression in eukaryotic cells in a controlled manner using a selectively-inducible expression vector such as the tetracycline system.

An additional object of present invention is to provide a method for the regulation of gene expression in eukaryotic cells in a controlled manner using an inducible vector such as the tetracycline system.

### SUMMARY OF THE INVENTION

The present invention is a selectively-inducible single-stranded DNA (ssDNA) expression library, a method for constructing the ssDNA expression library, a method for screening ssDNA expression library, and a method for identifying ssDNA molecules that switch bacterial gene(s) related to cell growth and toxin production and secretion on or off.

The method comprises a method for constructing a set of randomly ordered, fixed length oligodeoxynucleotide (ODN) strands and sub-cloning these ODNs into expression vectors constituted so that, when transformed into cells that are subsequently exposed to certain chemical environments, the cell reacts by expressing the individual ODN sequence programmed into the expression vector. Cells containing the instructions for an individual ODN are grown into colonies and each of the colonies is divided into control and experimental sets. When an experimental colony is exposed to the external chemical inducing the production of an ODN, the ODN is expressed and putatively alters cellular gene function, for instance, protein production, producing a different cell phenotype. If the phenotypic expression represents a desired end result, the control colony is treated to extract the DNA to determine the exact nucleotide sequence of the ODN that produced the phenotype in question.

This method is used to identify ODNs that, for instance, kill bacterial cells, thereby making it possible to identify new antibiotics against pathogenic bacteria and provide methods for identifying essential bacterial genes that can serve as additional targets for discovery of new antibiotics. The same methods and screening library is utilized to identify ssDNA molecules that switch bacteria gene(s) related to cell growth and toxin production and secretion on and/or off.

### BRIEF DESCRIPTION OF THE DRAWINGS

FIG. 1 is a schematic representation of a preferred embodiment of the expression vector pssXG constructed in accordance with the teachings of the present invention.

FIG. 2 shows the results of an assay for expression of HIS-tagged reverse transcriptase (RT) induced by tetracycline in E. coli. Bacterial cells were grown at 37° C. until OD 280 value of 0.5 and then 200 ng/ml of tetracycline was added to the cells and incubated for another eight hours. Cell Iysates were used for the assay, conducted in accordance with Silver, et al. (1993, Nucleic Acids Res. 21: 3593-3594). (1) without lysate, (2) without tetracycline induction, (3) with tetracycline induction. PCR amplification product is marked by an arrow.

FIG. 3 shows the results of an assay for expression of HIS-tagged reverse transcriptase (RT) induced by tetracycline in E. coli. Bacterial cells were grown at 37° C. until OD 280 value of 0.5 and then different amounts of tetracycline was added to the cells: (1) 1.0 ng/ml, (2) 0.1 mg/ml, (3) 1 mg/ml, (4) 10 mg/ml, (5) 100 mg/ml. Cell lysates were used for the assay, which detected RT expression by Western blotting. The RT band is marked with an arrow.

### DETAILED DESCRIPTION OF THE PREFERRED EMBODIMENT

Construction of Tetracycline-Inducible Prokaryotic ssDNA Expression Vector.

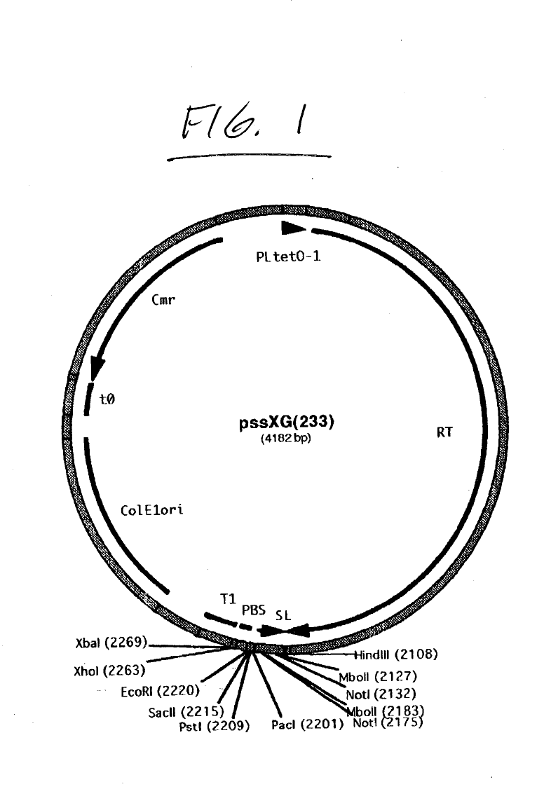

PCR amplification was carried out using pssXE, described in International Application No. PCT/US00/27381, which application is hereby incorporated into this specification in its entirety by this specific reference, as the template. DNA primers used in the PCR reaction, 5′NheIPvuIATG (5′-CTAGCTAGCTAGCGATCGAT-GGGACCAATGGGGCAG-3′) and 3′KpnI (5′-CGGGGTACCAGTATTCCCTGGTC-3′) were synthesized by Integrated DNA Technologies (Coralville, Iowa). The PCR amplified DNA fragment was double-digested with NheI and KpnI and then subcloned into the pssXE vector that was double-digested with the same enzymes. The replacement removes the sequence before the translation starting site (ATG), which is unnecessary for prokaryotic gene expression, while creating a new restriction enzyme site, PvuI. The newly created construct was digested with PvuI and XbaI. The PvuI-Xbal fragment contains all the essential elements for ssDNA production, including: 1) Mouse Moloney leukemia viral reverse transcriptase (MoMuLV RT) gene coding for a truncated but fully active RT (Tanase & Goff, PNAS, 2000, 85:1777-1781); 2) primer binding site (PBS) along with some flanking regions of the promoter that are essential for the reverse transcription initiation by MoMuLV RT (Shinnick, et al., Nature, 1981, 293:543-548); and 3) stem-loop structure designed for the termination of the reverse transcription reaction all as described in the above-incorporated International Application No. PCT/US00/27381. This DNA fragment was subcloned into the pPROTet.E 233 vector (BD Bioscience, Palo Alto, Calif.) and the newly created construct was designated as pssXG, shown in FIG. 1. However, the sequence of bacteria tRNAPro is different from mammalian tRNAPro, which was designed to bind with the PBS in mammalian cells. Because bacterial tRNAVal can be utilized as primer for RT, a new PBS was designed to replace the PBS used in the vector pssXE that is used for mammalian cells. The sequence of the novel PBS is: 5′TGGTGCGTCCGAG3′.

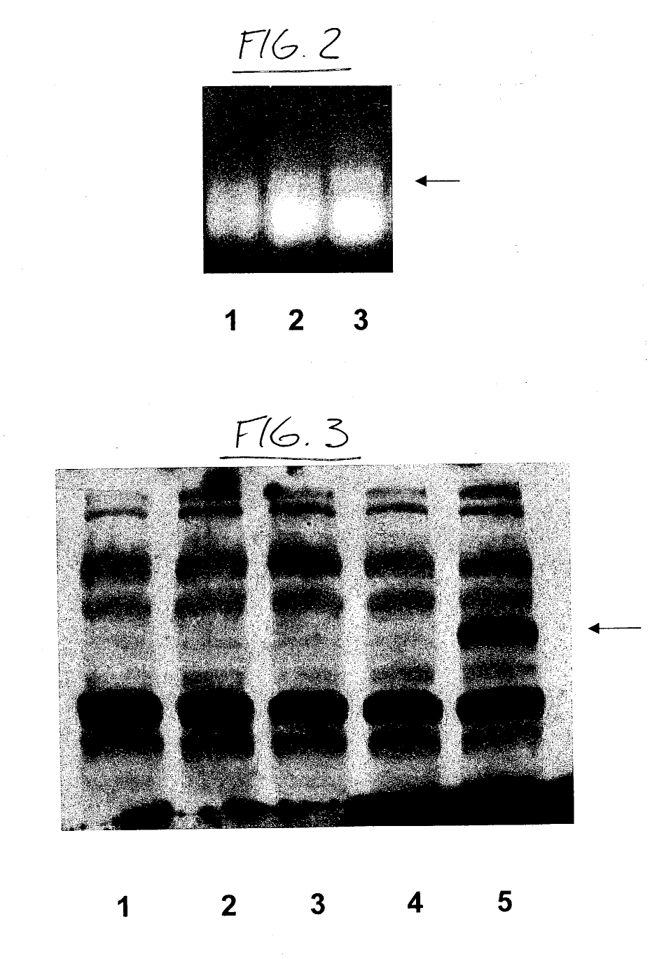

pPROTet.E233 is a tetracycline-inducible bacterial expression vector expressing fusion protein with 6×HN. It utilizes a novel promoter, P_{Ltet}O1, which is tightly repressed by the highly specific Tet repressor protein and induced in response to anhydrotetracycline (aTc), allowing control of induction over a wide range (anhydrotetracycline is a derivative of tetracycline that acts as a more potent inducer of PROTet.E Systems). The pssXG vector was transformed into the bacteria strain, DH5αPro (BD Bioscience, Palo Alto, Calif.) in the presence of 34 μg/ml choloramphenicol (Cm) and 50 μg/ml spectinomycin (spec). Spectinomycin is used to select for DH5αPro cells that carry transcription units encoding TetR (Lutz & Bujard, Nucleic Acids Res., 1997, 25:1203-1210). The DH5αPro cells express defined amounts of the Tet repressors. Cell lysates were prepared using B-PER II Bacterial Protein Extraction Reagent (Pierce, Rockford, Ill.) according to the manufacturer's instruction. Using the cell lysates, the expression of reverse transcriptase (RT) was confirmed by RT activity assay using cell lysates according to Silver, et al. (Nucleic Acids Res., 1993, 21:3593-3594) as shown in FIG. 2 and Western blotting using antibody against 6×HN (BD Bioscience, Palo Alto, Calif.) as shown in FIG. 3.

Construction of a Tetracycline-Inducible ssDNA or ODN Expression Library.

The library inserts were generated by annealing three ODNs, CY(SacII)-40, CTCTCACTCC(N)40ACTGTTGAAAGGC, CY(SacII)-L, CGGAGAGTGAGG and CY(SacII)-R, CTTTCAACAGT at the molar ratio of 1:20:20. Here, “N” represents any of the bases A, T, C, or G. There are thus 40-mer sequences randomly synthesized and represented as CY(SacII)-40 ODNs. All the ODNs were mixed and denatured at 95° C. for 3 min and then cooled down slowly to the room temperature over approximately 1 hr. Since CY(SacII)-L complements the left arm of CY(SacII)-40 while CY(SacII)-R complements the right arm of the same ODN, partial double-stranded ODNs are formed by the annealing process. The annealed ODN formed a partial double-stranded DNA and was filled in those remaining single-stranded Ns and blunt ended using the DNA Polymerase I, Large (Klenow) Fragment (New England Biolabs, Beverly, Mass.). The double-stranded DNA was then subcloned into newly created prokaryotic ssDNA expression vector designated pssXG and subsequently transformed into bacterial cells, DH5αPRO using electroporation.

ssDNA Expression Library Screening.

Since the ssDNA expression library was constructed based on a tetracycline inducible vector, bacterial cells containing the library were plated in duplicate in Luria broth (LB) plates in the presence or absence 100 μg/ml aTc. Colonies growing only in the absence of aTc and colonies plated with aTc were both isolated for examination of the status of the colonies and for further analysis. Plasmids from those isolated colonies were sequenced using 3′PROTet Seq primer (Bioscience, Palo Alto, Calif.) to identify the particular oligo(s) expressed.

Inhibition of Bacterial Growth By DNA Enzyme Targeted to FtsZ

Cell division is critical for bacterial survival; bacteria such as Escherichia coli normally divide by binary fission, producing two daughter cells of equal size, each containing a nucleoid. FtsZ is an essential gene for bacterial division and viability. The division process starts with the localization of FtsZ to the center of the mother cell and formation of a septal structure, the Z ring. Other essential cell division proteins are then recruited to the Z ring. Deletion and mutation of the ftsZ gene blocks cell division at an early stage, showing promise as a target for developing a new antibiotic agent.

Because of their ability to bind and cleave any target RNA at purine/pyrimmidine junctions, DNA enzymes are capable of interfering with gene expression as described in the above-incorporated International Application No. PCT/US00/27381. 10-23 DNA enzyme cleavage sites are plentiful in most biological substrates and thus provide a host of opportunities to achieve maximum cleavage efficiency. Based on the predicted secondary structure of FtsZ mRNA, a 10-23 DNA enzyme targeted against a GU site at position 880 was designed. The sequence of this 31 nt DNA enzyme is 5′GTTTCGAAGGCTAGCTACAACGATCATCCAG3′, with the predicted free energy 21.3 kcal/mol. This sequence was subcloned into the pssXG inducible vector.

The bacterial expression system was tested for production of RNA-cleaving DNA enzyme targeted to FtsZ mRNA. E. Coli cells engineered to produce regulatory protein Tet repressor were transformed with the plasmid pssXG designed to generate 31 nt DNA enzyme capable of cleaving FtsZ mRNA. The plasmid without the DNA enzyme sequence was used as negative control. Cell growth rate was assayed by measuring OD600. After induction by aTc, reduced bacterial growth rate up to 50% was observed in the transformed cells. No repression was seen when the negative control was tested.

Those skilled in the art who have the benefit of this disclosure will recognize that certain changes can be made to the component parts of the apparatus of the present invention without changing the manner in which those parts function to achieve their intended result. All such changes, and others which will be clear to those skilled in the art from this description of the preferred embodiments of the invention, are intended to fall within the scope of the following, non-limiting claims.

1. An oligodeoxynucleotide (ODN) library comprising a collection of randomly sequenced oligodeoxynucleotides of specific length, each said ODN capable of interacting with a target genomic DNA, mRNA or protein and inserted into a single stranded DNA expression vector with the specific calling sequence for each ODN embedded within said expression vector capable of being introduced into a target cell and capable of being induced by exposure to a chemcial agent to produce a plurality of individual ODNs from said library, one or more of said individual ODN having the capability to interact with genomic DNA, mRNA or protein with observable result.

2. A process for identifying and isolating an oligodeoxynucleotide comprising the steps of:
utilizing the ODN library of claim 1 to express a plurality of copies of an individual ODN in a target cell;
growing the target cells into a colony of cells;
dividing the colony into paired colonies;
exposing one of the paired colonies to a chemical agent capable of inducing expression of the ODN coded into an expression vector contained by the cells of the exposed colony, causing the expressed ODN to interact with genomic DNA, mRNA or a protein resulting in down regulation of a protein;
observing the result in said exposed cells; and
sequencing the DNA of the cells of the unexposed colony to identify the sequence of the library ODN whose interaction caused the down regulation of a target protein.

3. The method of claim 2 wherein said cells are bacteria strain DH5α(Pro.

4. The plasmid pssXG.

5. The plasmid of claim 4 comprising a PBS having the sequence 5′TGGTGCGTCCGAG3′ (SEQ ID NO: 3).

6. A cell having the plasmid of claim 4 transformed therein.

7. A prokaryotic cell having the plasmid of claim 4 transformed therein.

8. The plasmid of claim 4 comprising a sequence coding for in vivo expression of a single-stranded DNA enzyme targeted to the bacterial FtsZ gene.

9. The plasmid of claim 8 wherein the single-stranded DNA enzyme is specific for a GU site at position 880 of the bacterial FtsZ gene.

10. A cell having the plasmid of claim 8 transformed therein.


SEQUENCE LISTING (8 sequences)

SEQ ID NO: 1 - DNA, 36 bp, Artificial Sequence
  Description of Artificial Sequence Synthetic primer
ctagctagctagcgatcgatgggaccaatggggcag

SEQ ID NO: 2 - DNA, 23 bp, Artificial Sequence
  Description of Artificial Sequence Synthetic primer
cggggtaccagtattccctggtc

SEQ ID NO: 3 - DNA, 13 bp, Artificial Sequence
  Description of Artificial Sequence Synthetic primer
tggtgcgtccgag

SEQ ID NO: 4 - protein, 12 aa, Artificial Sequence
  Description of Artificial Sequence Synthetic fusion protein tag
HNHNHNHNHNHN

SEQ ID NO: 5 - DNA, 63 bp, Artificial Sequence
  Description of Artificial Sequence Synthetic primer
ctctcactccnnnnnnnnnnnnnnnnnnnnnnnnnnnnnnnnnnnnnnnnactgttgaaaggc

SEQ ID NO: 6 - DNA, 12 bp, Artificial Sequence
  Description of Artificial Sequence Synthetic primer
cggagagtgagg

SEQ ID NO: 7 - DNA, 11 bp, Artificial Sequence
  Description of Artificial Sequence Synthetic primer
ctttcaacagt

SEQ ID NO: 8 - DNA, 31 bp, Artificial Sequence
  Description of Artificial Sequence Synthetic primer
gtttcgaaggctagctacaacgatcatccag